(19) United States
(12) Patent Application Publication
(10) Pub. No.: US 2024/0358551 A1
(43) Pub. Date: Oct. 31, 2024

(54) ROBOTIC ASSISTED PROCEDURES

(71) Applicant: Auris Health, Inc., Redwood City, CA (US)

(72) Inventors: Jeffery B. Alvarez, Redwood City, CA (US); Greg Kintz, Santa Cruz, CA (US); David Mintz, Los Altos Hills, CA (US); Serena Wong, Mountain View, CA (US)

(21) Appl. No.: 18/656,491

(22) Filed: May 6, 2024

Related U.S. Application Data
(63) Continuation of application No. 16/986,064, filed on Aug. 5, 2020, now Pat. No. 11,974,948.
Application No. 16/986,064 is a continuation of application No. 14/301,871, filed on Jun. 11, 2014, now Pat. No. 10,744,035.
(60) Provisional application No. 61/833,835, filed on Jun. 11, 2013.

(51) Int. Cl. A61F 9/008; A61B 34/30; A61B 34/37; A61F 9/007

(52) U.S. Cl. CPC A61F 9/008; A61B 34/30; A61B 34/37; A61F 9/00736; A61F 2009/00851; A61F 2009/0087; A61F 2009/00887; A61F 2009/00889

(57) ABSTRACT

Techniques for facilitating the removal of material from an anatomical structure with a robotic system are discussed herein. For example, the robotic system can include a first robotic component configured to control movement of a first device and a second robotic component configured to control movement of a second device. The first device and the second device can be controlled within a chamber of the anatomical structure to remove material from the chamber. For instance, the first device can be controlled to emit energy to break up the material into pieces and the second device can be controlled to remove the pieces from the chamber. In some cases, the first device can be controlled to gradually vary a parameter associated with the first device between a first value and a second value.

### CROSS REFERENCE TO RELATED APPLICATION(S)

This application is a continuation of U.S. application Ser. No. 16/986,064, filed Aug. 5, 2020. which is a continuation of U.S. application Ser. No. 14/301,871, filed Jun. 11, 2014 (U.S. Pat. No. 10,744,035), which claims the benefit of U.S. Provisional Application No. 61/833,835, filed Jun. 11, 2013, the entire contents of which are incorporated herein by reference.

### BACKGROUND OF THE INVENTION

### 1. Field of the Invention

The field of the present application generally pertains to medical devices. More particularly, the field of the invention generally pertains to an apparatus, system, and method for robotic assisted cataract surgery.

A “cataract” is a clouding of the lens in the eye that affects vision. Most people develop cataracts due to aging. The condition is not uncommon; it is estimated more than half of all Americans will either have a cataract or have had cataract surgery by age 80.

FIG. 1 is a diagram of the human eye, included for background. The major features of the eye 100 comprise the cornea 101, the anterior chamber 102, the iris 103, the lens capsule 104, the lens 105, the vitreous 106, the retina 107, and the sclera 108. The lens capsule 104 has an anterior surface 109 bordering the anterior chamber 102 and a posterior surface 110 bordering the vitreous 106. Most relevant to cataracts, the lens 105 within the lens capsule 104 is comprised of a nucleus 111 and cortex 112.

As shown in FIG. 1, the lens 105 within the eye 100 lies behind the iris 103. In principle, it works much like a camera lens by focusing light onto the retina 107 at the back of the eye 100 where an image is recorded. The lens 105 also adjusts the focus of the eye 100, allowing it to focus on objects both near and far.

The lens 105 within the eye 100 contains protein that is precisely arranged to keep the lens 105 clear and allow light to pass through it. As the eye 100 ages, the protein in the lens 105 may clump together to form a “cataract”. Over time, the cataract may grow larger and obscure a larger portion of the lens 105, making it harder for one to see.

Age-related cataracts affect vision in two ways. The clumps of protein forming the cataract may reduce the sharpness of the image reaching the retina 107. The clouding may become severe enough to cause blurred vision. The lens 105 may also slowly change to a yellowish/brownish tint. As the lens 105 ages, objects that once appeared clear may gradually appear to have a brownish tint. While the amount of tinting may be small at first, increased tinting over time may make it more difficult to read and perform other routine activities.

Surgery is currently the only real treatment for cataracts. Each year, ophthalmologists in the United States perform over three million cataract surgeries. The vast majority of cataracts are removed using a procedure called extracapsular cataract extraction (ECCE). ECCE traditionally comprises of several steps. Incisions must first be made to the cornea 101 in order to introduce surgical instruments into the anterior chamber 102. Through the incisions in the cornea 101 and the space of the anterior chamber 102, the surgeon may remove the anterior face of the lens capsule 109 in order to access the lens underneath 105. This phase of the surgery, known as capsulorhexis, is often the most difficult procedure in ECCE. Having gained access to the lens through capsulorhexis, a small amount of fluid may be injected into the exposed lens capsule 104 to improve access and maneuverability of the lens 105. This phase of the surgery is known as hydrodissection to the skilled artisan.

After loosening the lens, it must be extracted. Traditionally, this phase of the procedure involved manual extraction of the lens through a large (usually 10-12 mm) incision made in the cornea 101 or sclera 108. Modern ECCE is usually performed using a microsurgical technique called phacoemulsification, whereby the cataract is emulsified with an ultrasonic hand piece and then suctioned out of the eye through incisions in the cornea 101.

A phacoemulsification tool may be an ultrasonic hand piece with a titanium or steel needle. The tip of the needle may vibrate at an ultrasonic frequency to sculpt and emulsify the cataract while a pump aspirates particles through the tip. In some circumstances, a second fine steel instrument called a “chopper” may be used to access the cataract from a side port to help with “chopping” the nucleus 111 into smaller pieces. Once broken into numerous pieces, each piece of the cataract is emulsified and aspirated out of the eye 100 with suction.

As the nucleus 111 often contains the hardest portion of the cataract. emulsification of the nucleus 111 makes it easier to aspirate the particles. In contrast, the softer outer material from the lens cortex 112 may be removed using only aspiration. After removing the lens material from the eye 100, an intraocular lens implant (IOL) may be placed into the remaining lens capsule 104 to complete the procedure.

One variation on phacoemulsification is sculpting and emulsifying the lens 105 using lasers rather than ultrasonic energy. In particular, femtosecond laser-based cataract surgery is rapidly emerging as a potential technology that allows for improved cornea incision formation and fragmentation of the cataract.

Phacoemulsification and laser-based emulsification, however, still have their shortcomings. Phacoemulsification requires the use of tools that propagate ultrasound energy along the length of the tool, from a proximal transducer to a distal tip. The propagation leads to the transmission of ultrasound energy along the tool to other tissues proximal to the eye 100. Ultrasound tools also generate more heat than would be desirable for a procedure in the eye 100. In addition, the mechanical requirements of propagating the ultrasound wave along the length of the tool often make it rigid and difficult to steer around corners or bends.

Laser-based tools have their own drawbacks. Presently, manually controlled lasers require careful, precise movement since they can easily generate unwanted heat in the eye 100. Laser fibers in the tool are also fragile, and thus easily damaged when attempting to navigate tight corners. Both limitations increase surgery time and raise safety concerns.

An alternative to conventional laser systems, femtosecond laser systems have their advantages and drawbacks as well. Femtosecond laser systems may be used to create entry sites through the cornea 101 and sclera 108 into the eye 100, as well as to remove the anterior face of the capsule 104. Femtosecond laser energy may be focused within the lens nucleus 111 itself, and used to “pre-chop” the lens nucleus 111 into a number of pieces that can then be easily removed with aspiration. Femtosecond lasers, however, can only fragment the central portion of the lens 105 because the iris 103 blocks the peripheral portion of the lens 105. Thus, use of another emulsification technology—ultrasound or conventional laser—is still necessary to fracture and remove the peripheral portion of the cataract in lens 105, extending total procedure time. Furthermore, femtosecond laser systems are also expensive and costly to operate and maintain.

Therefore, it would be beneficial to have a new method, apparatus, and system for performing all the phases of cataract surgery with improved precision control and reduced procedure time.

### SUMMARY OF THE INVENTION

In general, the present invention provides medical methods, apparatus and systems. Exemplary embodiments provide a method, apparatus, and system for robotic assisted cataract surgery. In one aspect, the present invention provides for a system for robotic ophthalmologic surgical procedures comprising an instrument drive mechanism comprising an instrument interface coupled to a robotic tool, wherein the robotic tool comprises a robotic tool tip, and a vision system configured to detect a position of at least one of an anatomical structure of a patient such as an eye of a patient and the robotic tool tip, and generate a signal in response to changes to the position, wherein the instrument drive mechanism is configured to manipulate the robotic tool tip in response to the signal.

In related systems, the system may also comprise a robot master system configured to receive commands from a user and manipulate the robotic tip in response to a change in position of the anatomical structure. In some embodiments, the instrument drive mechanism is a robotic arm. The robotic tool tip will often comprise one or more of a laser device, an ultrasonic device, an irrigation device, and an aspiration device. The instrument interface may be configured to couple to a plurality of different robotic tools such as at least one of a laser device, an ultrasonic device, an irrigation device and an aspiration device. A laser fiber of the laser device may be configured to extend beyond the length of the robotic tool tip. In embodiments with an irrigation device and an aspiration device in the robotic tool tip, the irrigation device and the aspiration device may be jointly configured to maintain a material volume inside an enclosed operative space of the anatomic structure. This may include maintaining a volumetric pressure within the eye of the patient. The vision system may comprises one or more of a white light imaging device, a structured light imaging device, or an optical coherence topographical device. The robotic tool tip may comprise a reflective marker that is configured to be detected by the vision system, or the robotic tool tip may be configured to deploy an umbrella-like structure.

In another aspect, the present invention provides for a method of laser emulsification during cataract surgery comprising detecting a position of at least one of an anatomical structure of a patient such as an eye of a patient and a robotic tool tip using a vision system, generating a signal in response to the position, and manipulating the robotic tool tip using a robotic instrument drive mechanism in response to the signal which may be a change in the position, wherein the robotic tool tip is coupled to a robotic tool that is coupled to an instrument interface of the robotic instrument drive mechanism. The robotic tool may comprise at least one of a laser device, an ultrasound device, an irrigation device, and an aspiration device. A laser fiber of the laser device may be configured to extend beyond the length of the robotic tool tip. The irrigation device and the aspiration device may be jointly configured to maintain a material volume inside an enclosed operative space of the eye of the patient. In related systems, robotically manipulating the robotic tool tip includes robotically maneuvering the robotic tool tip relative to the eye of the patient, robotically changing the pulse repetition rate of the laser device of the robotic tool tip, and robotically changing the pulse energy of the laser device of the robotic tool tip. The instrument drive mechanism may be configured to manipulate the robotic tool in response to a change in the position. The instrument drive mechanism may be configured to manipulate the robotic tool in response to commands from a user.

These and other embodiments are described in further detail in the following description related to the appended drawing figures.

### BRIEF DESCRIPTION OF THE DRAWINGS

The invention will be described, by way of example, and with reference to the accompanying diagrammatic drawings, in which:

FIG. 1 is a diagram of the human eye, included for background;

FIG. 2 is an illustration of a robotic surgery system with laser system according to an embodiment of the invention;

FIG. 3 is an illustration of the use of two tools with separate functionality for laser-based emulsification, irrigation, and aspiration, according to an embodiment of the present invention;

FIG. 4 is an illustration of the use of two tools with separate functionality for laser-based emulsification, according to an embodiment of the present invention;

FIG. 5 is an illustration of a cross-sectional view of a simple laser fiber tool for robotic control according to an embodiment of the present invention;

FIGS. 6A, 6B, and 6C are illustrations of a laser fiber suction tube with retractable laser fiber according to an embodiment of the present invention;

FIGS. 7A, 7B, 7C, 7D, and 7E are illustrations of tool 601 with retractable laser fiber 608 in various stages of extension and retraction of laser fiber 608;

FIGS. 8A and 8B are illustrations of various views of robotically-assisted lasing paths for laser-based emulsification, according to certain embodiments of the present invention;

FIGS. 9A and 9B are illustrations of robotic laser tools that execute a preprogrammed lasing pattern for laser-based emulsification and aspiration, according to certain embodiments of the present invention;

FIG. 10 is an illustration of a robotic laser tool that executes a preprogrammed spiral lasing pattern for laser-based emulsification, according to an embodiment of the present invention;

FIGS. 11A, 11B, 11C, and 11D are illustrations and diagrams that show a lasing protocol that manipulates the pulse energy, tool tip speed, and pulse repetition rate based on the progress of the tool along a predetermined tool path according to an embodiment of the present invention;

FIG. 12 is a structural illustration of an apparatus with a laser fiber and a means of aspiration, according to an embodiment of the present invention;

FIGS. 13A, 13B, 13C, and 13D are block diagrams of systems with real time flush and/or aspiration control, according to certain embodiments of the present invention;

FIG. 14 is a diagram of a method of registration for tools and anatomical objects within the anterior chamber of an eye, according to certain embodiments of the present invention;

FIG. 15 is a diagram of a method of registration for non-linear tools and anatomical objects within a lens capsule, according to certain embodiments of the present invention;

FIG. 16 is an illustration of a robotic laser tool that executes a preprogrammed spiral lasing pattern with the aid of a vision or OCT system, according to an embodiment of the present invention;

FIGS. 17A, 17B, 17C, and 17D are illustrations and charts showing the operation of a robotic laser device where a lasing protocol manipulates the pulse repetition rate based on real-time information retrieved by a vision or OCT system, according to an embodiment of the present invention;

FIGS. 18A and 18B are illustrations of tool tip articulation according to certain embodiments of the present invention;

FIG. 19 is an illustration of a cross-sectional view of a one-way bending laser probe, according to an embodiment of the present invention;

FIG. 20 is an illustration of a cross-sectional view of a concentric tube laser probe with OCT, according to an embodiment of the present invention;

FIG. 21 is an illustration of a cross-sectional view of a four-way articulating tool, according to an embodiment of the present invention;

FIGS. 22A and FIGS. 22B1-22B3 are illustrations of top-sectional views of robotically controlled capsulorhexis procedures according to certain embodiments of the present invention;

FIG. 23 is an illustration of a robotic apparatus with an instrument drive mechanism to couple tools to a robotic system, according to an embodiment of the present invention; and

FIG. 24 is an illustration of a robotic apparatus with a plurality of instrument drive mechanisms and interfaces, according to an embodiment of the present invention.

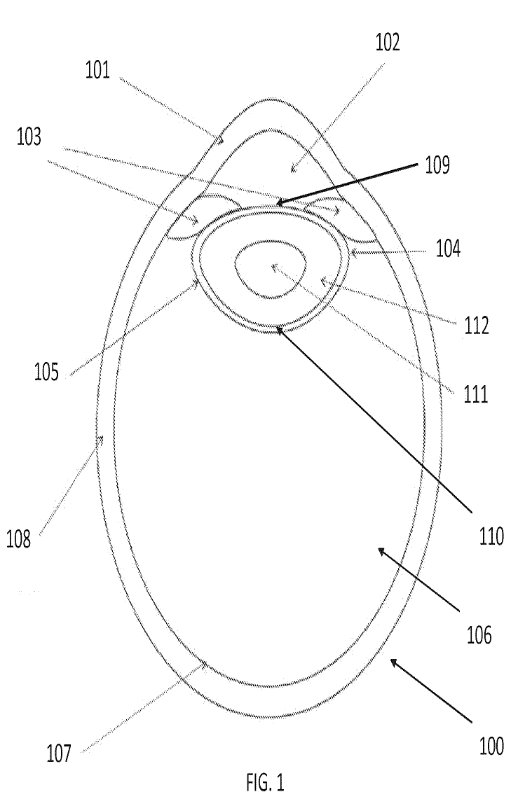

### DETAILED DESCRIPTION OF THE INVENTION

Although certain preferred embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in tum, in a manner that may be helpful in understanding certain embodiments; however, the order of description should not be construed to imply that these operations are order dependent. Additionally, the structures, systems, and/or devices described herein may be embodied as integrated components or as separate components. Accordingly, the methods and systems of the present invention are not limited to cataract surgery and other ophthalmologic applications.

### Bimanual Approach

The present invention relates to a robotic device to assist surgical procedures in ophthalmology, particularly cataract surgery. In some embodiments, a plurality of robotic devices may be used. In embodiments with two robots, each robot may hold its own laser tool in a dual-tool or “bimanual approach.” This approach allows for greater flexibility, as tools may be interchanged, positions may be adjusted, and approaches may be altered to allow for improved access to the operating area. For example, in some embodiments, the robotic arms may move in different patterns to speed up the removal of the cataract. In some embodiments, the robotic arms could move synchronously to treat the same area. Alternatively, both tools could be pointed towards the same general location, while a first tool could be angled from the top down and the other tool angled to undercut the portions of the lens cut by the first tool.

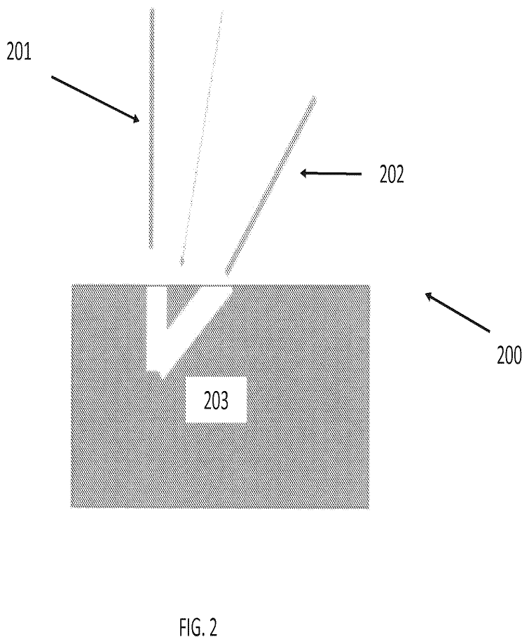

FIG. 2 illustrates a robotic surgery system with laser system according to an embodiment of the invention. As shown in FIG. 2, robotic surgery system 200 comprises a first tool 201 and a second tool 202. First tool 201 and second tool 202 may comprise at least one of the following to facilitate removal of cataract material from a sample lens capsule 203: lasers, optical coherence tomography (OCT) sensors, imaging systems, video systems, location sensors, flush devices, aspiration devices, and robotic articulation control.

In one embodiment, tool 201 includes a laser and a device with flush and aspiration capabilities. In one embodiment, tool 201 may be able to break up the cataract with the laser in small precise regions due to the strong absorption of the laser light by the cataract material, or water, mesh, or any thermal or mechanical effect. In some embodiments, the laser light in tool 201 may be altered to “undercut” the larger pieces of cataract material, i.e., use small cuts to remove large pieces.

In some embodiments, a laser in tool 201 may be coupled to the cataract though an optical fiber to a tool in the cataract. In other embodiments, a laser may be coupled through free space optics. In some embodiments, a laser may be emitted at the tip of tool 201. In some embodiments, the pulse energy, repetition rate, and pulse duration of the laser in tool 201 may be controlled in real-time. In controlling these parameters, a user of tool 201 may alter the extent and speed of cataract material removal.

Techniques that incorporate use of a computer-controlled robotic arm with preprogrammed patterns allow for precise control and motion of tool 201 and tool 202. Precise control and constant motion of the tools may allow a user of the tools to optimize the volumetric rate of removal of the cataract material using tool 201 and tool 202.

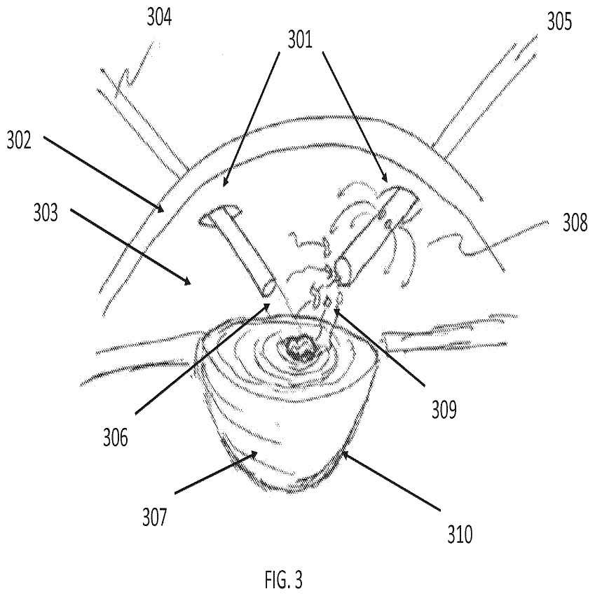

FIG. 3 illustrates an apparatus using two tools for laser-based emulsification in an eye, according to an embodiment of the present invention. In FIG. 3, a first tool 304 and a second tool 305 have separate and distinct functionalities as they operate within the anterior chamber 303 through incisions 301 in the cornea 302 in the eye 300. First tool 304 may emit laser radiation 306 for emulsifying cataract material in the lens 307. Second tool 305 may have flush capabilities 308 and aspiration capabilities 309 to extract the cataract material and maintain the integrity of the anterior chamber 303 and lens capsule 310.

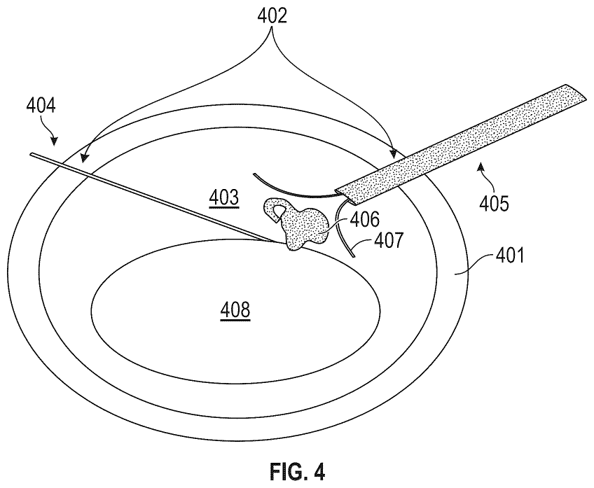

FIG. 4 illustrates an apparatus using two tools for laser-based emulsification, according to an embodiment of the present invention. In FIG. 4, a first tool 404 with a laser fiber and a second tool 405 with a hollow tube operate in the anterior chamber 403 through incisions 402 in the cornea 401. Laser radiation from first tool 404 may be used to break up the cataract in the lens 408 during laser-based emulsification. However, the resulting debris plume 406 caused by the breaking up of the cataract could potentially spray and damage the endothelial cells of the lens anterior chamber 403. Accordingly, the hollow tube in second tool 405 may deploy a membrane “umbrella” 407 to shield the endothelial cells of anterior chamber 403 during the emulsification process. Upon completion, umbrella 407 may be retracted back into the hollow tube in second tool 405 through incisions 402 in the cornea 401. In some embodiments, membrane umbrella 407 may be pre-shaped by spines created from nitinol.

### Laser Tools

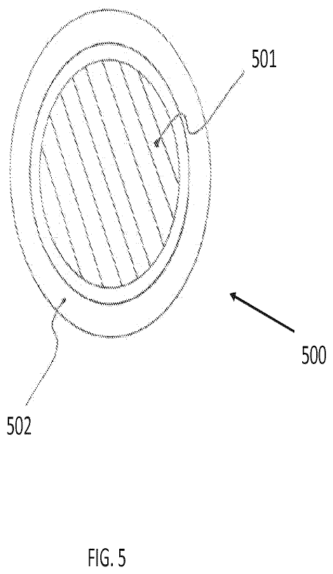

FIG. 5 is a schematic of a simple laser fiber tool for robotic control in accordance with an embodiment of the present invention. As shown in the cross-sectional view of FIG. 5, tool 500 may include a laser fiber 501 and outer tube 502. In some embodiments, the laser signal and energy may be conveyed to the tip of the tool via a photonic waveguide, a set of mirrors, or a set of mirrors and lenses. In some embodiments, the space between laser fiber 501 and outer tube 502 may be used to conduct flush and aspiration means.

Embodiments of the present invention may incorporate lasers with a wavelength near the water absorption peak at 2 μm, 3 μm, 4.5 μm, 6 μm, or 10 μm. Alternatively, a laser near the protein absorption peak at 280 nm may also be used to remove cataract material.

In some embodiments, tools may also contain more than one laser fiber. This could be used to increase the area that is treated or create lesions that are particular shapes (for example, three fibers in a row could produce a longer more knife-like cut).

During the operation of the laser, the tip of the laser tool may be located some distance away from the anterior portion of the capsule or in contact with the capsule. In the case of the laser tool being in contact with the capsule, the tip of the tool could be shaped to provide maximum cutting effect. Depending on the embodiment, the shapes of the laser tool tip can be flat, round, tapered to a point, or a combination of the flat, round and tapered shapes.

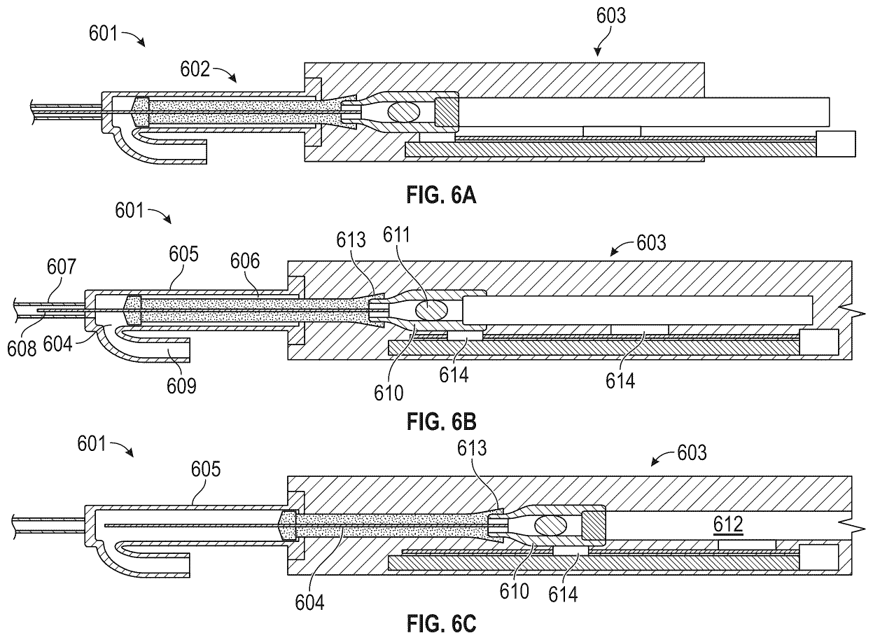

FIG. 6 illustrates a laser fiber suction tube with retractable laser fiber in accordance with an embodiment of the present invention. FIG. 6A is a general diagram of tool 601, which contains both a disposable portion 602 and a reusable portion 603.

FIG. 6B is a detailed diagram of the components within tool 601 where the plunger 604 is extended into a syringe cylinder 605, which is synonymous with disposable portion 602. Syringe cylinder 605 is proximally connected to reusable portion 603 to allow for the plunger 604 to extend into the plunger support shaft 606. Syringe cylinder 605 is distally connected to a suction tube 607 which may sheath a retractable laser fiber 608. Syringe cylinder 605 may also provide access 609 for aspirational means to suction tube 607. Within reusable portion 603, plunger 604 may be coupled to a lens coupling housing 610, a lens 611 within lens coupling housing 610, and a trunk fiber 612 through a plunger snap fitting 813. Plunger 604 may thus be extended into syringe cylinder 605 or retracted back to reusable portion 603 based on the movement of a controlled slider 614.

FIG. 6C is a detailed diagram of the components within tool 601 where plunger 604 is recessed into the reusable portion 603. As shown in FIG. 6C, when controlled slider 614 is retracted, trunk fiber 612 and lens coupling housing 610 are also retracted. Accordingly, plunger 604, which is coupled to lens coupling housing 610 through plunger snap fitting 613, is recessed from syringe cylinder 605.

During the emulsification and aspiration of the lens cataract material, the posterior portion of the capsule is at risk of being damaged from the laser radiation and the aspiration force. For example, if portions of the lens capsule membrane are sucked into the aspiration tube, the posterior portion may be stressed and torn. This increases the risk of intrusion of the vitreous fluid into the anterior portion of the eye, which can cause infections and other eye diseases. To minimize that possibility, in some embodiments, the laser fiber can be extended beyond the end of the suction tube to act as a probe when the laser energy is turned off. The use of the laser fiber as a probe prevents the suction tube from approaching the capsular membrane and damaging it. The shape of the laser fiber can be optimized to minimize damage to the membrane. Examples of the shaped tips include rounded or circular tips.

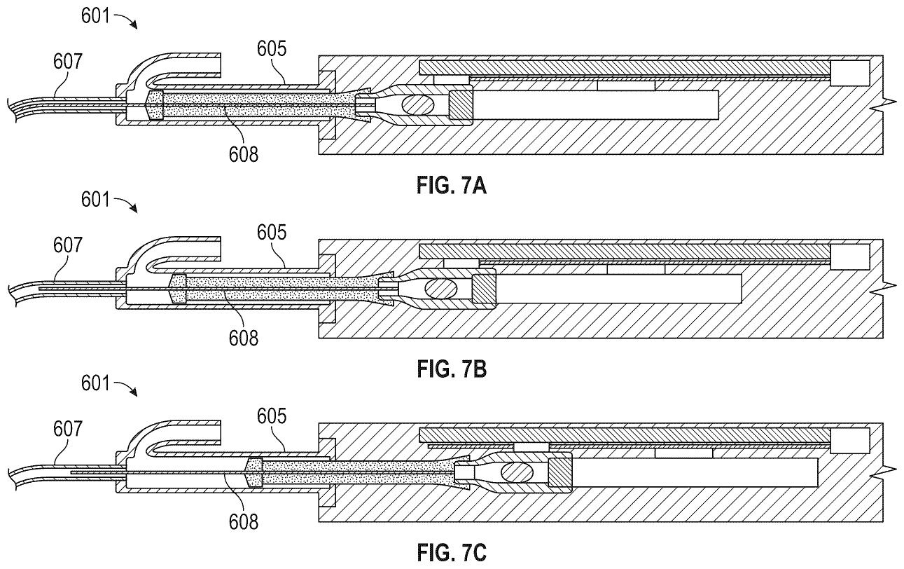

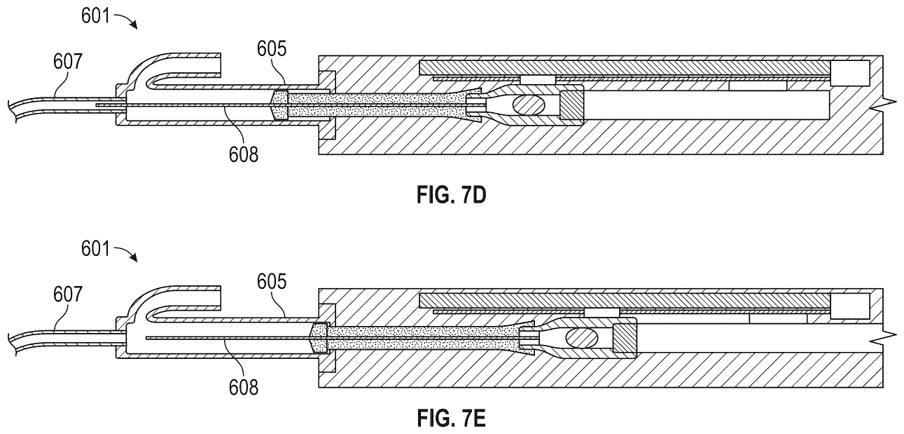

FIG. 7 shows tool 601 with retractable laser fiber 608 in various stages of extension and retraction. In FIG. 7A, the laser fiber 608 is extended beyond the end of the suction tube 607 for purposes discussed earlier. In FIGS. 7B, 7C, 7D, the laser fiber 608 is shown to be retracted such that the end of the laser fiber sits in the suction tube 607. In FIG. 7E, the laser fiber is shown to be retracted such that the end of the laser fiber sits in the syringe cylinder 605.

In some embodiments of the present invention, robotic control over preprogrammed patterns of lasing paths may be used to emulsify and aspirate the cataract from the lens capsule. In some embodiments, preprogrammed patterns may control the motion of a tool as well as the laser, flush, and aspiration parameters in order to optimize the removal of lens material. Compared to a manual technique, use of a computer-controlled robotic arm with preprogrammed patterns allows more precise control over the tool tip, generating more accurate patterns and more consistent motion that optimize the volumetric rate of removal during emulsification.

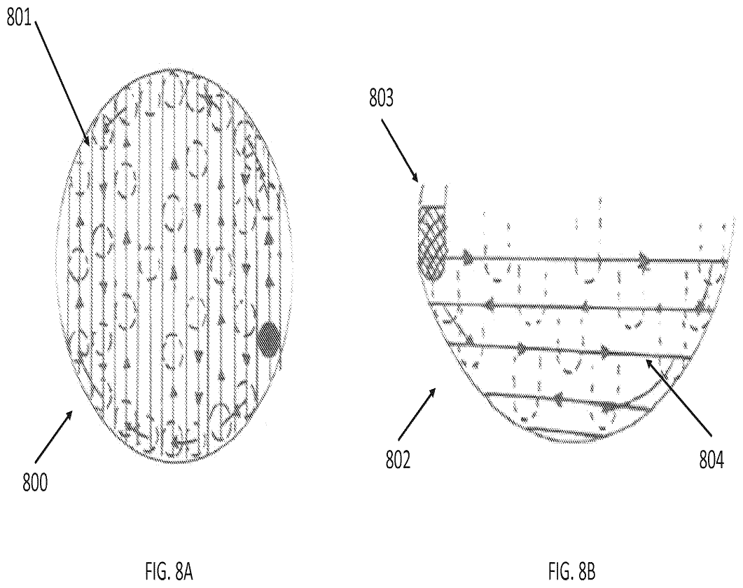

FIG. 8 illustrates different views of robotically-assisted lasing paths for emulsification, according to some embodiments of the present invention. FIG. 8A shows a lasing path 801 with parallel paths in the lens material 800 from the perspective of the anterior chamber and anterior portion of the lens capsule. Using a side-perspective, FIG. 8B shows the depth of a parallel lasing path 804 of the tool tip 803 in the lens material 802. In some embodiments, the emulsification process is at least partially enabled by a cavitation bubble that is created from steam generated by laser energy from a laser tool and fluid from an irrigation tool. The resulting shockwave from a steam-based cavitation bubble results in a larger laser effective zone. In some embodiments, the cavitation bubble may be based on different thermal, mechanical, and shock waves.

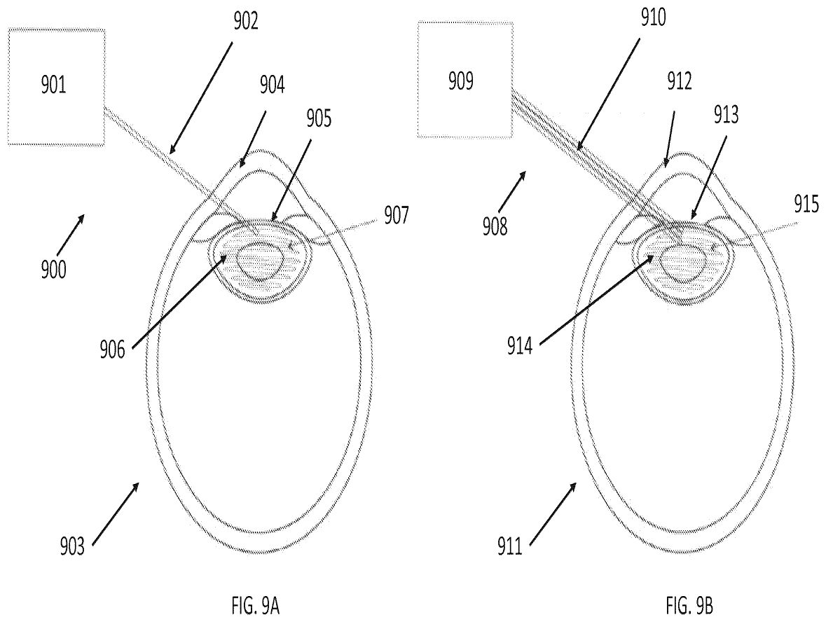

FIG. 9 illustrates an embodiment of the present invention where a preprogrammed emulsifying lasing pattern may be executed either with a simple laser tool (FIG. 9A), or a laser tool with integrated flush and aspiration means (FIG. 9B). FIG. 9A shows an apparatus 900 comprising a robot arm 901 and laser-based tool tip 902 as it follows a preprogrammed lasing pattern 907 inside the lens 906 through incisions in the cornea 904 and the lens capsule 905 of an eye 903. FIG. 9B shows an apparatus 908 comprising a robot arm 909 and tool tip 910 with laser and integrated flush and aspiration capabilities as it follows a preprogrammed lasing pattern 915 inside the lens 914 through incisions in the cornea 912 and the lens capsule 913 of an eye 911.

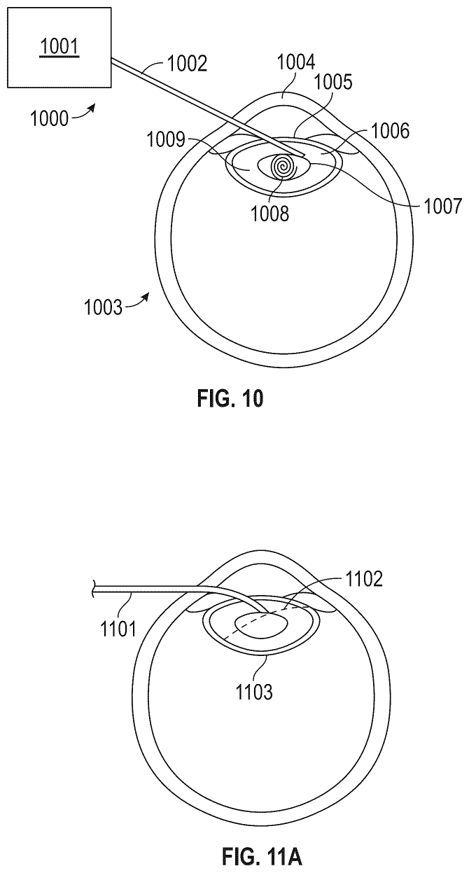

FIG. 10 illustrates an embodiment of the present invention executing a preprogrammed emulsifying lasing pattern that starts in the center of the eye and spirals out, effectively taking the nucleus of the eye out while leaving the cortex in place. The spiral lasing pattern may be executed by an apparatus 1000 with a robot arm 1001 controlling a laser tool 1002 that follows a spiral tool path 1007 through lens material 1006 within a lens capsule 1005 in eye 1003. Spiral tool path 1003 starts in the center of lens capsule 1005 to affect the nucleus 1008 and terminates to avoid the cortex 1009. The spiral lasing pattern affords more precise targeting of the hard cataract in the nucleus 1008 while also avoiding the cortex 1009 in order to minimize the potential damage to delicate membranes associated with the lens capsule 1005.

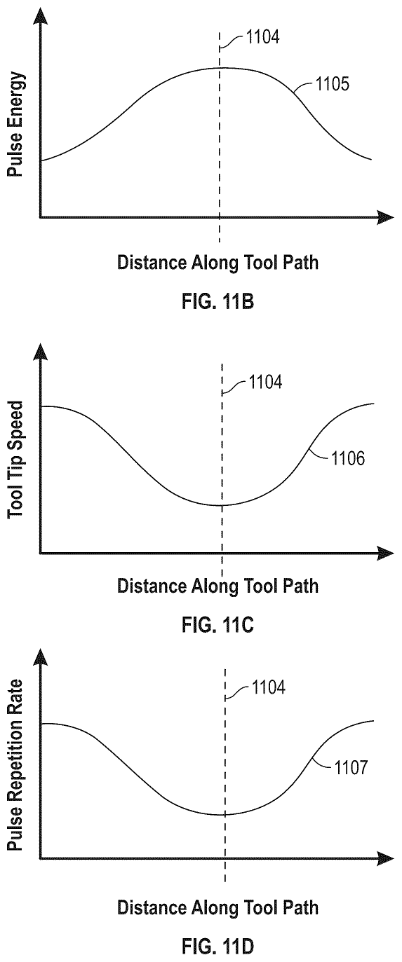

FIG. 11 provides several diagrams and charts to illustrate a laser-based emulsification protocol that manipulates the pulse energy, tool tip speed, and pulse repetition rate based on the progress of the tool along a predetermined tool path in accordance with an embodiment of the present invention. FIG. 11A illustrates an embodiment of the present invention where the emulsification tool tip 1101 travels along a simple tool path 1102 within a lens capsule 1103.

FIG. 11B provides a chart detailing how the laser pulse energy varies depending on the tool tip distance along the tool path. As shown by line 1105, the preprogrammed protocol increases the laser pulse energy during the mid-point of the tool path, represented by a vertical dotted line 1104, where the laser is aimed at the nucleus, where the cataract is often hardest.

FIG. 11C provides a chart detailing how the tool tip speed varies depending on the tool tip distance along the tool path. As shown by line 1106, the preprogrammed protocol slows tool tip speed during the mid-point 1104 of the tool path where the laser is aimed at the nucleus, where the cataract is often hardest.

FIG. 11D provides a chart detailing how the pulse repetition rate varies depending on the tool tip distance along the tool path. As shown by line 1107, the preprogrammed protocol slows pulse repetition rate during the mid-point 1104 of the tool path where the laser is aimed at the nucleus, where the cataract is often hardest. In contrast, the pulse repetition rate may be increased at the beginning and end of the tool path, when the tool tip is directed to the cortex, thereby compensating for the speed and/or improved removal of the softer lens cortex materials. In some embodiments, lower pulse repetition rate leads to greater laser pulse energy.

In some embodiments, the spacing between the arms of the spiral may be varied depending on the size of debris to be removed by aspiration. For example, if larger pieces can be removed, the arms of the gaps between neighboring paths of the spiral may become wider. As less cutting is required to break up the cataract, expanding the distance between the arms of the spirals helps reduce the cutting time. This protocol likely necessitates robotic control because due to precise spacing within the lens capsule makes the protocol very difficult to accomplish with a purely manual technique.

In some embodiments, raster patterns may remove material in a systematic x-y-z, grid-like motion. In other embodiments, a radial pattern reminiscent of a typical quadrant chop-and-divide technique may be employed.

In some embodiments, an emulsification patterns may remove lens material from the anterior portion of the cortex, remove by lens material from the center of the nucleus, before removing the remainder of the lens material in other portions of the cortex.

### Flush and Aspiration Tools

In yet another embodiment, the robotic system includes real-time control of a flush and aspiration device to remove debris from the chamber. Robotically-controlled fluidic flush and aspiration tool may improve the precision and accuracy of the procedure to maintain the material volume in an enclosed operative space like the interior of an eye. In some embodiments, a tool with a laser device may also have flush and aspiration capabilities. In the alternative, a second tool may include a dedicated aspiration and flush tool.

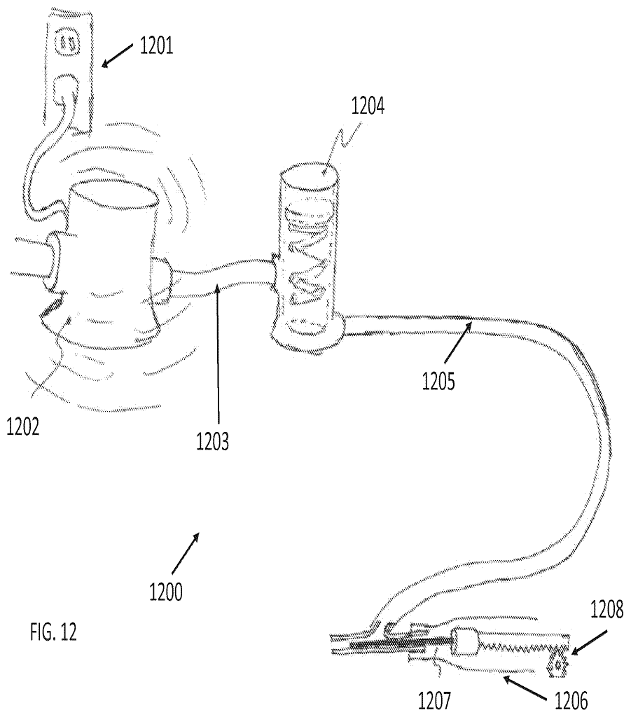

FIG. 12 illustrates structural components of an apparatus with a laser fiber and a means of aspiration, in accordance with an embodiment of the present invention. As shown in FIG. 12, apparatus 1200 comprises a power source 1201 that provides electricity to a vacuum pump 1202 coupled through a hose 1203 to a dampener 1204. The aspiration flow may be transferred from the dampener 1204 to a tool 1206 through a tube 1205. The dampener 1204 (represented by a plunger within a cylinder) moderates spikes and dips in the aspiration pressure in cases of air blockage or occlusion in tool 1206.

In addition to aspiration means, the tool may also comprise a laser fiber 1207 that may be extended or retracted using various mechanical means, such with axial motion from a gear 1208. In this particular embodiment, all aspirated cataract material passes near the laser fiber in the aspiration tube as it is being evacuated from the eye.

The size of the flush and aspiration channels also directly influence the size of the pieces of cataract that can be extracted from the lens capsule. Hence, the size of the channels could influence the emulsification patterns of the robotic procedure.

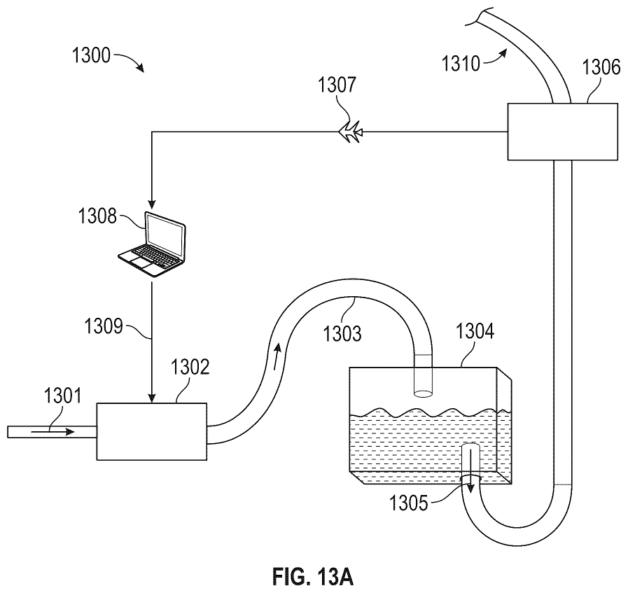

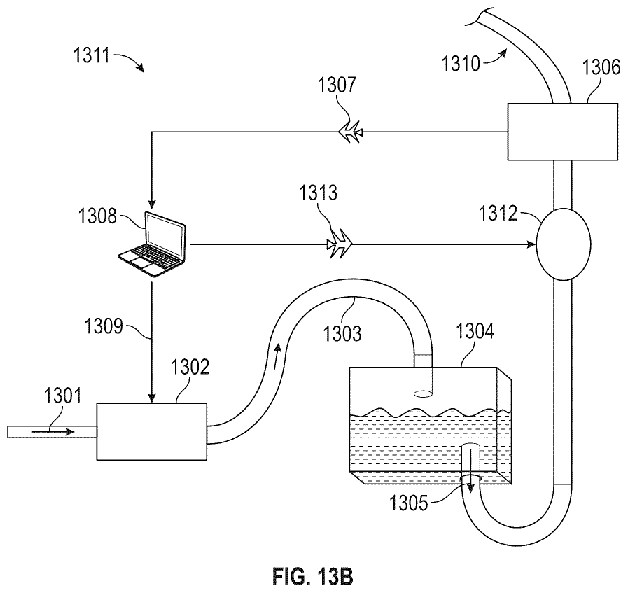

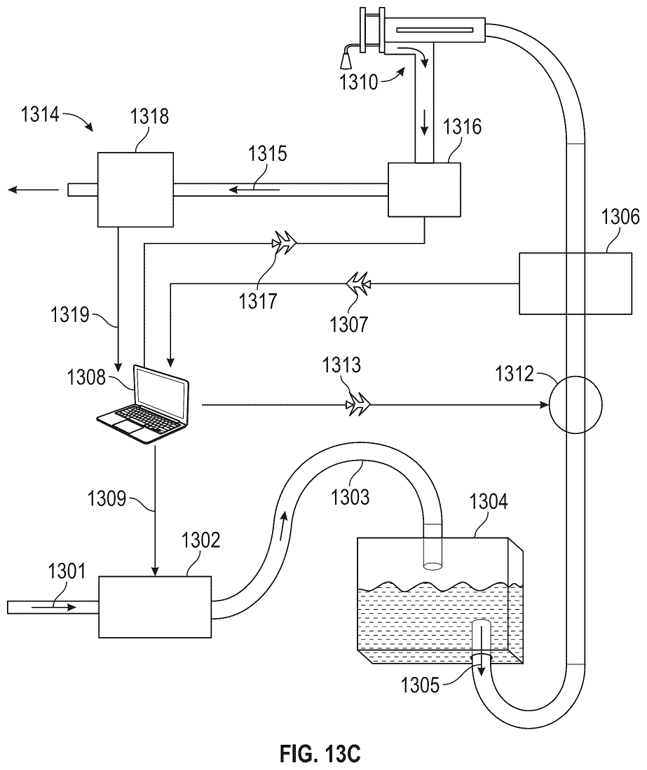

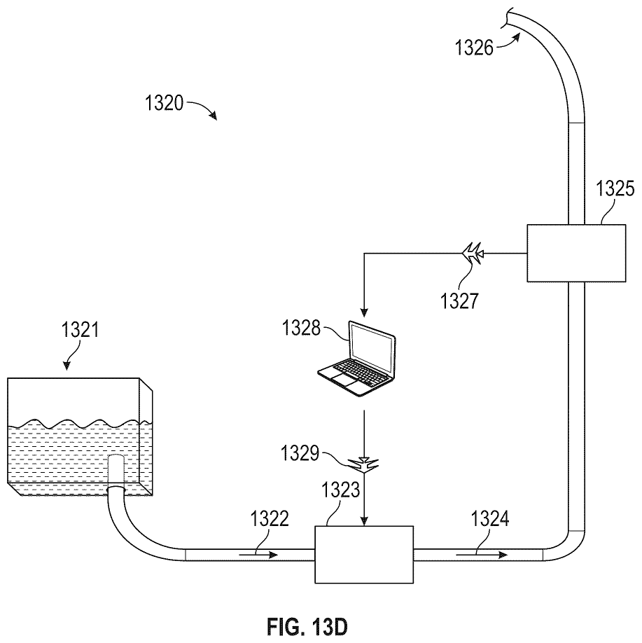

FIG. 13 illustrates block diagrams of several embodiments of the present invention with real-time flush and aspiration control. FIG. 13A illustrates a block diagram of apparatus 1300 with real-time flush control, in accordance with an embodiment of the present invention. In FIG. 13A, apparatus 1300 incorporates a pump 1302 controlled by a computer 1308, a pressure vessel 1304, and a flow rate meter 1306. In apparatus 1300, fluid 1301 is supplied to pump 1302. Pump 1302 directionally forwards the fluid to pressure vessel 1304 via a tube 1303. The fluid pumped into pressure vessel 1304 forces liquid that was originally resting in pressure vessel 1304 toward the flow rate meter 1306. The flow rate meter detects the velocity of the liquid 1305 prior to output 1310 from apparatus 1300. Information regarding the velocity of liquid flow is sent to computer 1308, which can send pressure signal 1309 to pump 1302. This creates a feedback loop; by communicating a flow rate feedback signal 1307 to computer 1308, computer 1308 can respond to the velocity of the exiting liquid 1305 by controlling the pump 1302 through pressure signal 1309. Hence, if the flow of liquid 1305 is too great, the flow of fluid from the pump 1302 through tube 1303 may be adjusted downward. Alternatively, if the flow of liquid 1305 is too low, the flow of fluid from pump 1302 through tube 1303 may be increased to exert more pressure on the liquid in pressure vessel 1304. The increased pressure in pressure vessel 1304 results in increased flow of liquid 1305. This feedback loop enables apparatus 1300 to moderate its liquid flow to a desired flush rate at output 1310.

FIG. 13B illustrates a block diagram of apparatus 1300 from FIG. 13A with the addition of a throttle valve to help facilitate the flow rate, in accordance with an embodiment of the present invention. In this embodiment, represented as apparatus 1311, a throttle valve 1312 receives the flow control signal 1313 from either a computer, central processing unit, microcontroller, ASIC, or other control circuitry which is represented as 1308. The throttle valve 1312 may further affect fluid flow by limiting (“throttling”) the fluid output from apparatus 1311.

FIG. 13C illustrates a block diagram of apparatus 1311 from FIG. 13B combined with an aspiration pump that removes material from the operative site, in accordance with an embodiment of the present invention. In this embodiment, represented by apparatus 1314, a desired aspiration flow 1315 may be obtained by controlling the aspiration pump 1316 with an aspiration control signal 1317 from computer 1308, which may also be a central processing unit, microcontroller, ASIC, or other control circuitry. In some embodiments, computer 1308 may control the pump 1302, the throttle valve 1312, and the aspiration pump 1316, using the pressure signal 1309, flow (flush) control signal 1313, and the aspiration control signal 1317 respectively. In some embodiments, the aspiration control signal 1317 may be generated by the computer 1308 in response to the aspiration flow rate feedback signal 1319 received from the aspiration flow rate meter 1318 and the flow rate feedback signal 1307 from tool flow rate meter 1306.

Thus, the tool flow rate meter 1306 and aspiration flow rate meter 1318 may be monitored to track the irrigation and aspiration flows in order to maintain an appropriate volume in an enclosed operative space like the interior of an eye of a patient. In this configuration, irrigation and aspiration flow may be moderated or interrupted in real-time using pump 1302, throttle valve 1312, and/or aspiration pump 1316. This may be done in the event that the aspiration path is unable to match the desired flow rate due to blockage, pinched tube, or other mechanical failure.

In some embodiments, the pressure signal 1309 may send a signal indicating fixed pressure to pump 1302. In those embodiments, irrigation (flush) flow may be maintained using flow (flush) control signal 1313 to control throttle valve 1312.

FIG. 13D illustrates a block diagram of an apparatus with real-time flush control where the fluid originates from a fluid reservoir, in accordance with an embodiment of the present invention. In FIG. 13D, fluid 1322 in apparatus 1320 originates from fluid reservoir 1321. Fluid 1322 enters pump 1323 and the pumped output fluid 1324 sent to flow rate meter 1325 prior to output 1326. Similar to the flow rate meter 1306 from FIGS. 13A, 13B, and 13C, the flow rate meter 1325 communicates a feedback signal 1327 about the readings from the flow rate meter 1325 to a computer, central processing unit, microcontroller, ASIC, or other control circuitry (represented as 1328) that controls the pump 1323 using a pump control signal 1329.

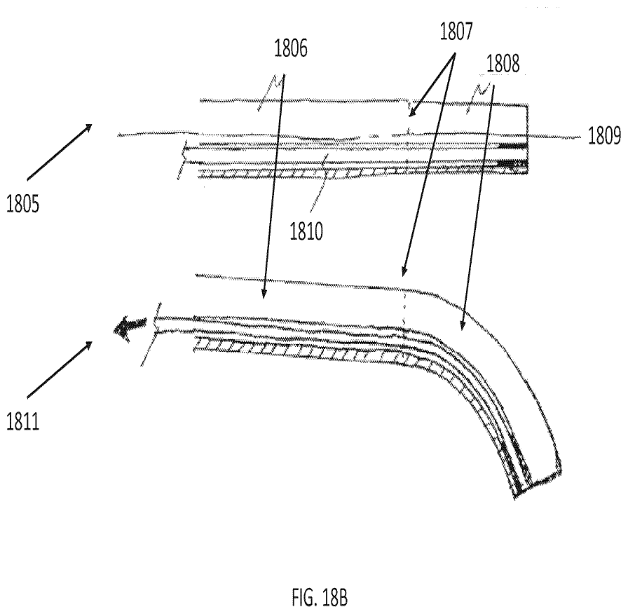

Unlike in FIGS. 18A, 18B, and 18C, the irrigation flow at output 1326 is directly-controlled by the control signal 1329 to pump 1323, rather than indirectly through a pressure vessel.

### Sensor & Imaging Tools

In one embodiment, a locational sensor or imaging technique may also be used to localize different portions of the cataract and the size of the cataract. Such locational sensors or imaging techniques may include 3D imaging, OCT, MRI, CT, Ultrasound, Intra-operative (OCT) or video systems with processing. In some embodiments, the tool itself may have an OCT device. In some embodiments, the tool may have multiple degree of freedom (dof) sensors, such as electromagnetic or fiber sensors.

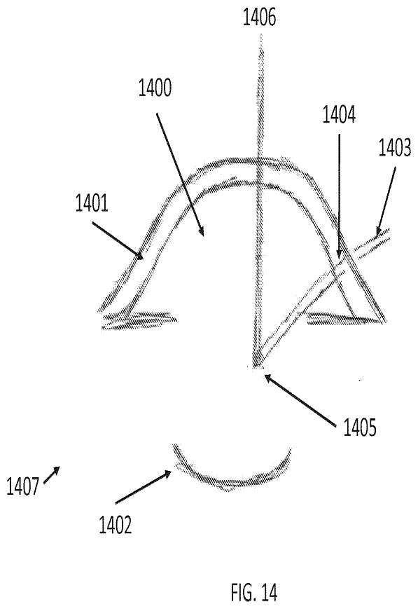

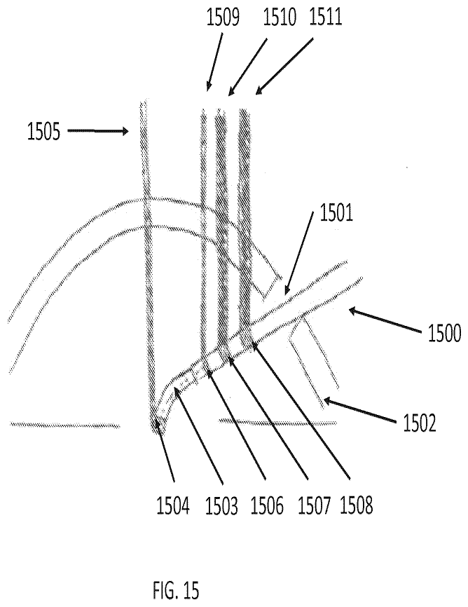

FIG. 14 illustrates a method of registration, the act of locating and tracking objects relative to a computer-generated map, for tools and anatomical objects within the anterior chamber 1400 of eye 1407, according to one embodiment of the present invention. Having inserted tool 1403 through an incision 1404 in the cornea 1401 in order to access a lens capsule 1402, the boundary and shape of the lens capsule 1402 may be captured to create a computer-generated map based on reflective portions of the lens capsule 1402.

In FIG. 14, OCT-based techniques may register the tool using the reflection signal 1406 from the reflective marker at the tool tip 1405 and the relative location of the tool base (not shown in FIG. 14). Using kinematics and reverse kinematics, the remaining portion of tool 1403, i.e., the shaft, may be computed based on the relative locations of the tool base and tool tip 1405. In OCT, the signals generate positional information in three dimensions. The reflective and refractive effects from the cornea and other optical surfaces may be compensated using optical de-warping. This form of registration, known as autocorrelation, starts by detecting signals at the tool tip based on the reflections from the reflective marks. Thus, using signals generated by this autocorrelation technique, the entire tool 1403 may be registered within the computer-generated map in Cartesian coordinates. Beyond OCT, other imaging modalities, including white light imaging and structured light imaging, can also be used to determine the location of the tool with respect to the anatomical features of the surgical field.

FIG. 15 illustrates a method of registration for non-linear tools and anatomy within a lens capsule, according to an embodiment of the present invention. In FIG. 15, a tool 1500 inserted through an incision 1501 in the cornea 1502 with a pre-bent tip 1503 with a reflective marker 1504 to assist autocorrelation of the tool tip 1503. The autocorrelation signal of the tool tip 1503 based on the reflective marker 1504 is represented as a vertical, orthogonal line 1505 emanating from the reflective marker 1504. Reflective markers 1506, 1507, and 1508 may be present at predetermined intervals along the length of the tool 1500. Using kinematics and autocorrelation techniques for markers 1506, 1507, and 1508, the entire tool 1500 may be precisely registered within a computer-generated map in Cartesian (x-, y-, and z-) coordinates.

Reflection signals 1509, 1510, and 1511 from markers 1505, 1506, and 1507 respectively may be used for registration.

Imaging techniques and sensors may also be used to optimize laser, flush, and aspiration parameters. For example, if it is detected that the tool tip 1503 is too close to anatomical structures, the laser power could be reduced to reduce the chance of injury.

Similarly, flush and aspiration pressure may be manipulated to facilitate removal of the cataract material.

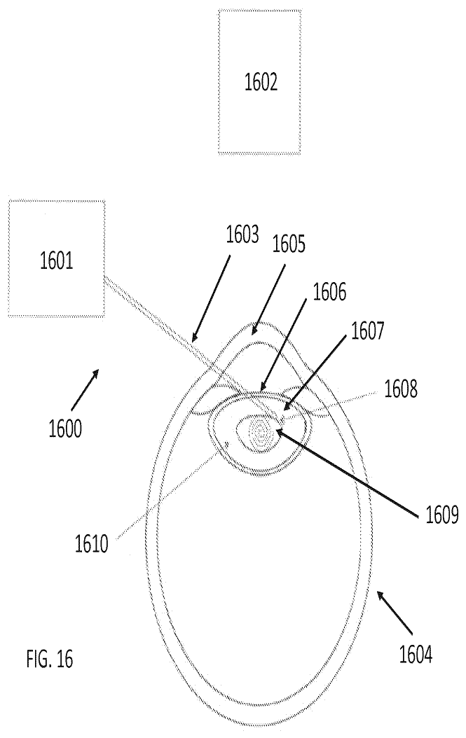

FIG. 16 illustrates an embodiment of the present invention executing a preprogrammed spiral lasing pattern with the aid of a vision, video or OCT system. In FIG. 16, a spiral tool path may be executed by a robotic system 1600 with a vision or OCT system 1602 and a robot arm 1601 that controls a laser tool 1603 that follows a tool path 1608 within the lens capsule 1606 in the eye 1604. Like FIG. 10, tool path 1608 starts in the center of the lens 1607 in the lens capsule 1606 in the nucleus 1609 and spirals outwards until it terminates near the cortex 1610.

In some embodiments, the imaging system can be traditional white light imaging and structured light imaging. In some embodiments, robotic controls sensitive to localization sensors and imaging may improve the safety of the tool and its ability to bound and calculate a pre-programmed lasing pattern to fit a particular patient profile.

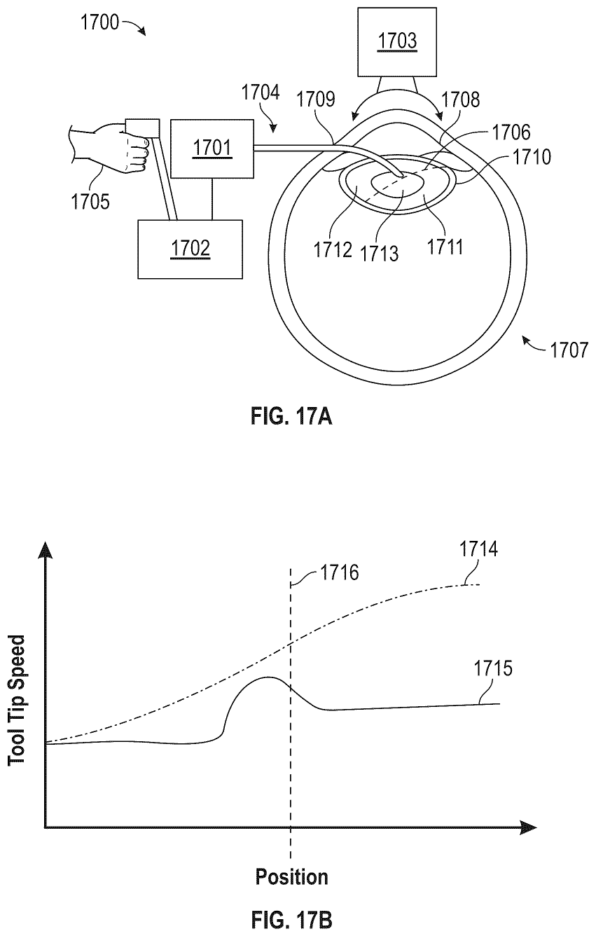

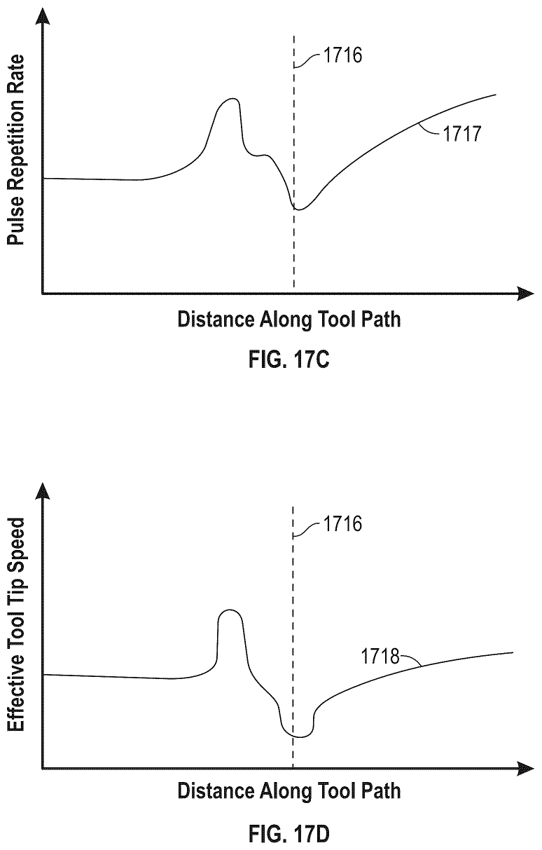

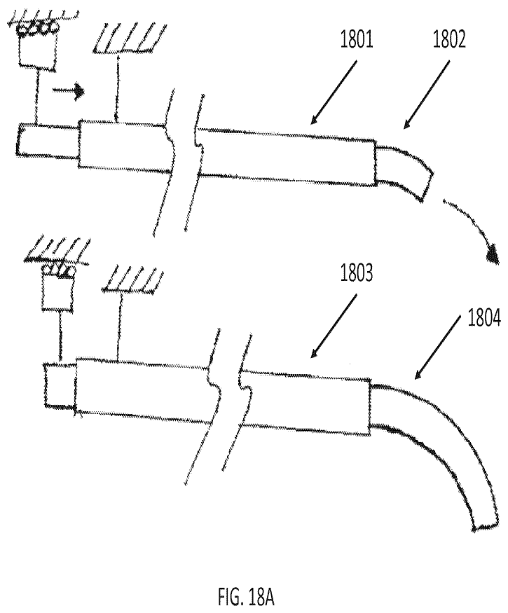

FIG. 17 provides several diagrams where a lasing protocol manipulates the pulse repetition rate based on real-time information retrieved by a vision or OCT system in accordance with an embodiment of the present invention. In FIG. 17A, apparatus 1700 controls a robot tool 1701 with tool tip 1704 in response to control commands from a doctor 1705 on a robot master system 1702 and a vision or OCT system 1703. Tool tip 1704 may be manipulated by doctor 1705 over a tool path 1706 through the lens 1711 in the lens capsule 1710, the nucleus 1713, and lens cortex 1712 in a patient's eye 1707.

FIG. 17B provides a chart detailing how the tool tip 1704 and the patient's eye 1707 may move as the doctor maneuvers tool tip 1704 through tool path 1706. In FIG. 17B, line 1714 illustrates how tool tip speed varies as doctor 1705 commands the tool tip 1704 to maneuver along tool path 1706. In this example, rather than maneuver the tool tip 1704 at a steady speed, doctor 1705 increasingly maneuvers tool tip 1704 at a faster rate as it moves along the tool path 1706. Line 1715 illustrates how the eye 1707 moves as tool tip 1704 moves along tool path 1706. In this example, the patient's eye 1707 does not remain stationary, but moves as tool tip 1704 progresses down tool path 1706. Indeed, eye 1707 shifts immediately prior to tool 1704 reaching the lens nucleus 1713, represented by vertical dotted line 1716.

FIG. 17C provides a chart detailing how apparatus 1700 compensates for variations in tool tip speed 1714 and eye movement 1715 by altering pulse repetition rate in the laser in tool tip 1704. In FIG. 17C, line 1717 illustrates how apparatus 1700 varies the rate of pulse repetition of laser in tool tip 1704 as it traverses tool path 1706. As shown by line 1717, near the beginning of the tool path 1706, apparatus 1700 rapidly increases the pulse repetition rate when eye 1706 moves to create more exposure to lens cortex 1709. Conversely, as shown by the intersection of lines 1716 and 1717, when the tool tip 1704 coincides with the nucleus 1713, the pulse repetition rate quickly drops to address where the cataract is often hardest. Similar to the embodiment in FIG. 11, lower pulse repetition rate leads to greater laser pulse energy in the system of FIG. 17.

FIG. 17D provides a chart detailing how changes to the pulse repetition rate represented by line 1718 results in an “effective” tool tip speed that varies as tool tip 1704 traverses tool path 1706. Line 1718 illustrates how varying the pulse repetition rate of the laser in tool tip 1704 results in an effective tool speed, i.e., equivalent tool tip speed assuming a consistent pulse repetition rate. Consequently, use of vision or OCT systems 1703 allows apparatus 1700 to compensate for inconsistent tool tip speed and eye movement by altering the pulse rate repetition rate automatically in real-time. Accordingly, altering the pulse repetition rate has the same effect altering the tool tip speed, shown by line 1718 in FIG. 17D.

### Tool Articulation

In some embodiments, the tool tip may sit in a robotically controlled articulating region. The articulation region may allow movement of the tip of the tool while avoiding motion in the rest of the tool. In some embodiments, the articulation region may include pre-bent tubes, pre-bent tubes recessed within straight or bent tubes, flexures with control wires, flexures fabricated with semiconductor fabrication technologies, and flexures with micro-motors and micro-gears. Use of a robotically controlled articulating tip minimizes the size of the incision in the lens capsule necessary to extract the cataract material. Hence, this is an important technology for capsulorhexis as will be discussed below.

An example of an articulating tool is an optical fiber encased in a pre-bent tube, where the pre-bent tube has a rigid, straight exterior tube. In some embodiments, the pre-bent tube can be retracted into the straight tube, creating a tool that can change from a bent to a straight configuration. The amount of retraction can be controlled robotically, allowing the bend on the tool to be synchronized with the tool pattern and or laser parameters. Use of a pre-bent tube does not limit the articulation means that can be used with the tool tip, other means include a flexure with one or more control wires.

In some embodiments, the present invention may include a robot for positioning the tip of the tool in space and optionally providing for angular degrees of freedom for adjusting the direction of the laser tool. FIGS. 18A and 18B illustrates tool tip articulation in accordance with certain embodiments of the present invention. FIG. 18A illustrates concentric tube articulation as the pre-bent tool tips 1802 and 1804 extend from tools 1801 and 1803. As shown in FIG. 18A, the pre-bent tool tips 1802 and 1804 exhibit greater deviation from a neutral axis as they are extended a greater distance from tools 1801 and 1803.

FIG. 18B illustrates a detailed view of the tool tip articulation from the perspective of the laser fiber within the tool. As shown in tool tip 1805, a shaft 1806 may have an articulation point 1807 and a bend section 1808 beyond the articulation point 1807. When unbent, the shaft 1806 and bend section 1808 remain aligned around a neutral axis 1809. Similarly, a laser fiber 1810 fixedly coupled to tool tip 1805 would remain aligned around neutral axis 1809 when unbent. As shown in tool tip 1811, however, when articulated or bent, bend section 1808 may articulate beyond articulation point 1807.

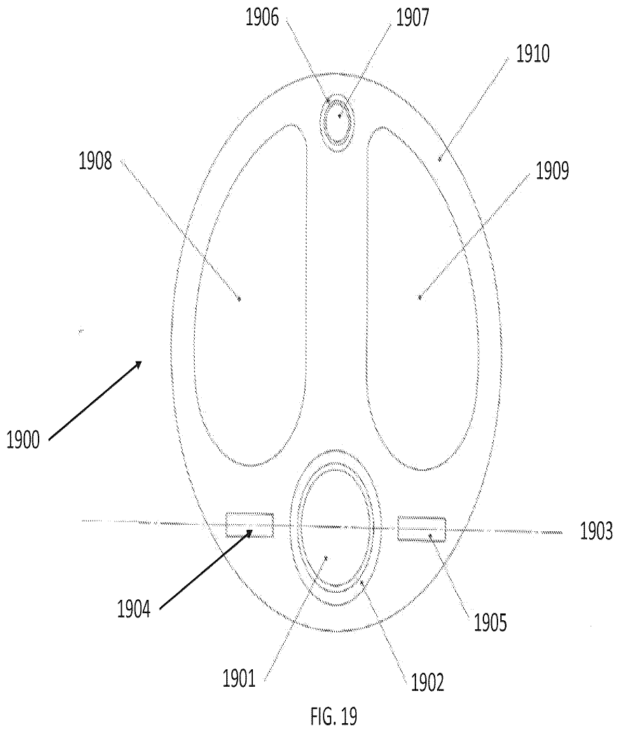

FIG. 19 illustrates a cross-sectional view of a one-way bending laser probe, according to an embodiment of the present invention. In this embodiment, the tool 1900 includes a laser fiber 1901 within a laser lumen 1902. The tool's imaginary neutral axis 1903 runs through the laser lumen and the structural members 1904, 1905. Also, the tool 1900 contains a control lumen 1906 with a control tendon 1907, a flush lumen 1908, and an aspiration lumen 1909. A flexible extrusion 1910 is also included.

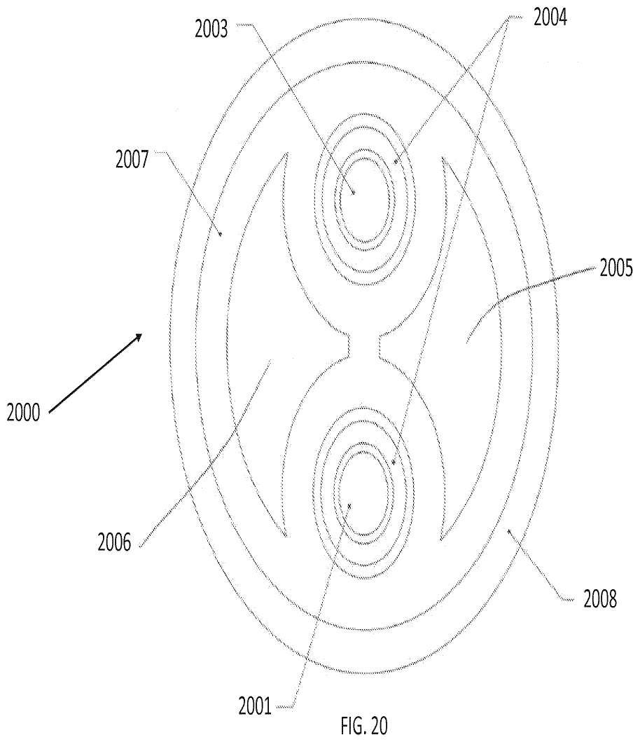

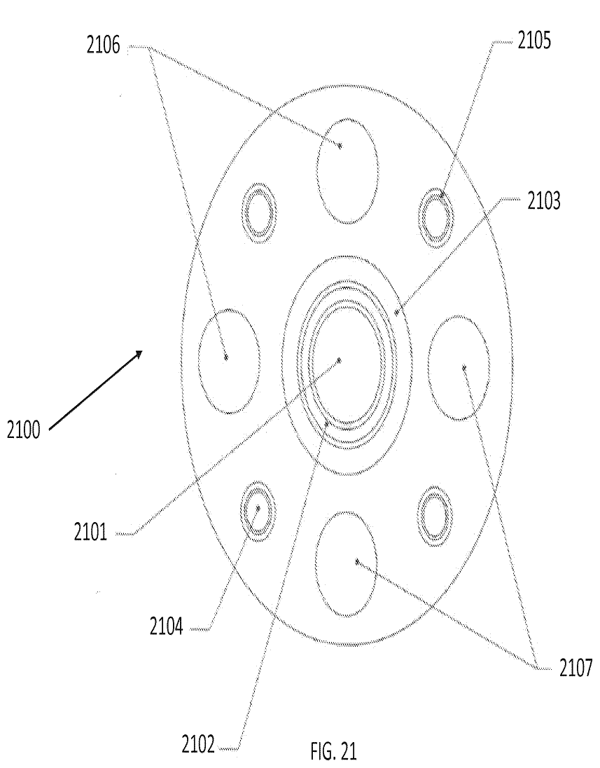

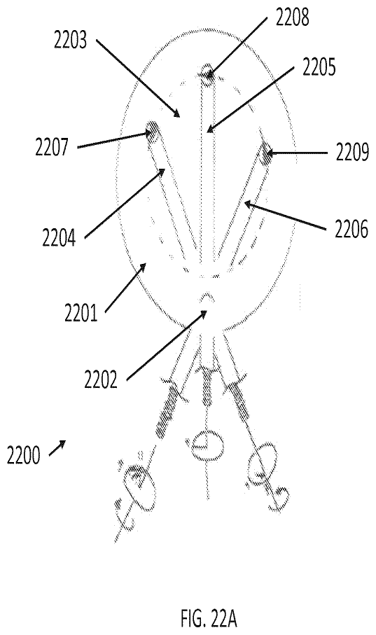

FIG. 20 illustrates a cross-sectional view of a concentric tube laser probe with OCT, according to an embodiment of the present invention. In this embodiment, the tool 2000 includes a laser fiber 2001 surrounded by a polyimide liner 2002. Also, the tool 2000 contains an OCT fiber 2003 surrounded by a polyimide liner 2004, a flush lumen 2005, and an aspiration lumen 2006. A flexible multi-lumen extrusion 2007 and a formed deflectable tube 2008 surrounds the tool 2000 is also included.

FIG. 21 illustrates a cross-sectional view of a four-way articulating tool, according to an embodiment of the present invention. In this embodiment, the articulating tool 2100 includes a laser fiber 2101 surrounded by a fiber jacket 2102, enclosed within a structural member 2103. As a four-way articulation tool, the tool 2100 contains a four control tendons, such as 2104, each enclosed by a tube member, such as 2105. The tool also contains a plurality of flush lumens 2106 and aspiration lumens 2107.

### Applications for Capsulorhexis

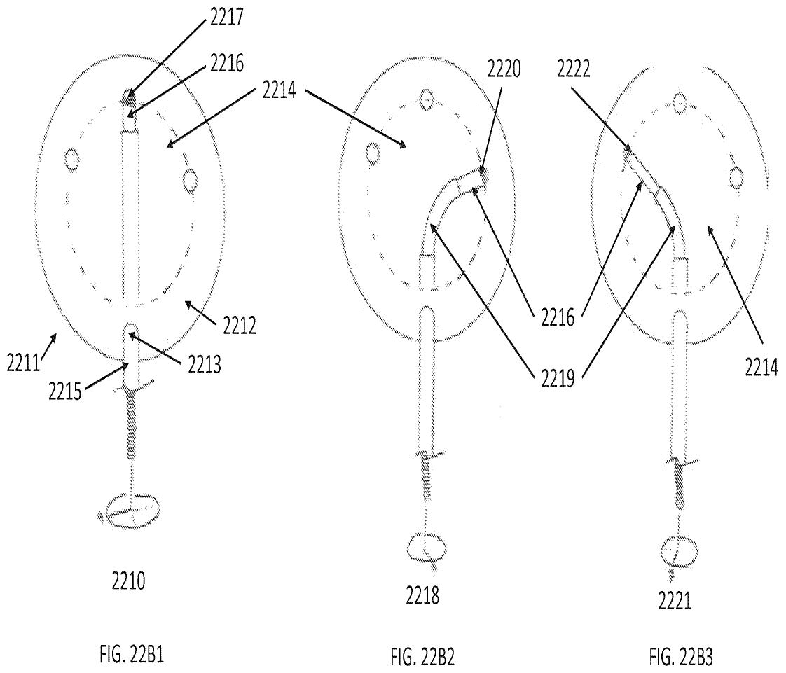

FIG. 22A and FIGS. 22B1-22B3 illustrate top-sectional views of a robotically controlled capsulorhexis procedure, according to two embodiments of the present invention. FIG. 22A specifically illustrates an embodiment of the invention where the tool tip (represented in several angles as 2204, 2205, 2206) may access different portions of the surface of the lens capsule 2203 by pivoting the tool tip from an incision 2202 in the cornea 2201 of the eye 2200. In FIG. 22A, the tool tip may be pivoted into a leftward position 2204 to make incisions on the left section 2207 of surface of the lens capsule 2203. In other circumstances, the tool tip may be directed straight (shown as 2205) at a diametrically opposed portion 2208 of the surface of the lens capsule 2203. In addition, the tool tip may be pivoted to access the right section 2209 of lens capsule 2203. A laser device may be coupled to the end of the tool tip in angles 2204, 2205, and 2206. In some embodiments, pivoting at incision 2202 allows for two part motion of the tool, including yaw and roll movement.

FIGS. 22B1-22B3 illustrate top-sectional view of a robotically controlled capsulorhexis procedure using an embodiment of the present invention where shaped tubes, axially translatable to vary articulation angle, enable complex distal tip motion so that the tool tip may access different portions of the surface of a lens capsule.

As shown in scenario 2210, tool 2215 may be directed orthogonally towards the surface of lens capsule 2214 through an incision 2213 in the cornea 2212 in eye 2211. Having penetrated cornea 2212, tool tip 2216 may be extended from tool 2215 to target a diametrically opposed region 2217 on the surface of the lens capsule 2214. In scenario 2218, tube 2219 and tool tip 2216 may be manipulated to maneuver tool tip 2216 to target a right-side region 2220 of the surface of lens capsule 2214. Similarly, in scenario 2221, tube 2219 and tool tip 2216 may be manipulated to maneuver tool tip 2216 to target a left-side region 2222 of the surface of lens capsule 2214.

In some embodiments, a laser device may be coupled to the end of the tool tip 2216 in scenarios 2210, 2218, and 2221. In some embodiments, tube 2219 is constructed from pre-bent tubes from nitinol. In some embodiments, tube 2219 may be articulated using either pull wires, tendons, or cables. In some embodiments, the construction of tool 2215, tube 2219, and tool tip 2216 allows for roll motion, irrigation, and aspiration in both tube 2219 and tool tip 2216.

### Instrument Drive Mechanisms

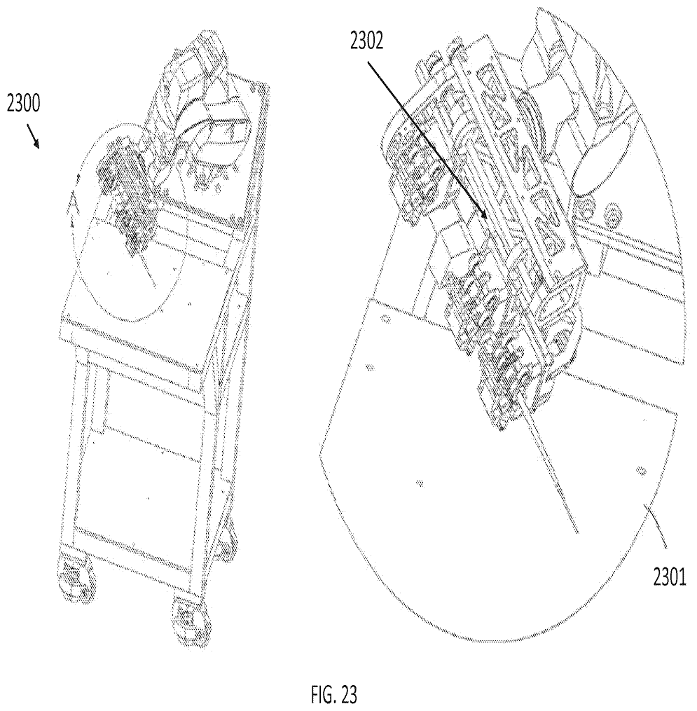

FIG. 23 illustrates an apparatus with an instrument drive mechanism to couple the tools described above to a robotic system, in accordance with an embodiment of the present invention. An instrument drive mechanism is a piece of mechanized equipment capable of driving an instrument or tool. In FIG. 23, the instrument drive mechanism 2302 is shown in a close view 2301 of the entire apparatus 2300. For this embodiment, the apparatus may be a robotic system with a proprietary instrument drive mechanism, such as the da Vinci® Surgical System by Intuitive Surgical, Incorporated, the Magellan™ Robotic System by Hansen Medical, Incorporated, the Micron by Carnegie Mellon University, or the Steady-Hand Eye Robot by John Hopkins University.

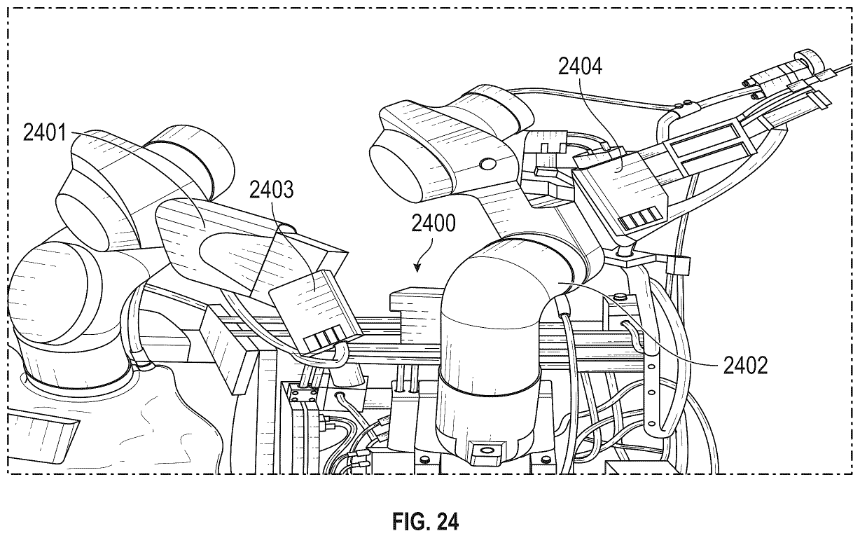

FIG. 24 illustrates a robotic apparatus with a plurality of instrument drive mechanisms and interfaces, in accordance with an embodiment of the present invention. In FIG. 24, the apparatus 2400 comprises two robotic arms, i.e., instrument drive mechanisms, identified as 2401 and 2402. Robotic arms 2401 and 2402 each have an individual instrument interface, shown as 2203 and 2204 respectively. Instrument interfaces 2203 and 2204 may each couple to a catheter-like tool, such as those discussed earlier.

In several embodiments of the present invention, the previously described tools—catheters, laser, OCT, flush and aspirations tools—may be controlled and coupled to the instrument drive mechanisms discussed in FIGS. 23 and 24 though instrument interfaces. Similarly, the components discussed with respect to FIGS. 13A, 13B, 13C, and 13D, such as flow rate meter, computer, feedback loop, pump, and pressure vessel, may be coupled to the robotic arm or instrument drive mechanisms discussed in FIGS. 23 and 24.

The present invention is not limited to embodiments using the aforementioned systems and the associated instrument drive mechanisms. One skilled in the art would appreciate modifications to facilitate coupling to different robotic arm configurations.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein. Elements or components shown with any embodiment herein are exemplary for the specific embodiment and may be used on or in combination with other embodiments disclosed herein. While the invention is susceptible to various modifications and alternative forms, specific examples thereof have been shown in the drawings and are herein described in detail. The invention is not limited, however, to the particular forms or methods disclosed, but to the contrary, covers all modifications, equivalents and alternatives thereof.

1-20. (canceled)

21. A method comprising:
inserting a first tool into a chamber of an anatomical structure, the first tool comprising a first device and configured to be coupled to a first robotic arm;
inserting a second tool into the chamber of the anatomical structure, the second tool comprising a second device and configured to be coupled to a second robotic arm;
emitting energy from the first device at material within the chamber;
aspirating pieces of the material from the chamber via the second device; and
gradually varying a parameter of the first tool associated with the energy between a first value and a second value based on a distance that a tip of the first tool has traveled along a tool path.

22. The method of claim 21, wherein the material is associated with the anatomical structure.

23. The method of claim 21, wherein the material includes an undesired clump of material in the chamber.

24. The method of claim 21, wherein the gradually varying is based on movement of the anatomical structure.

25. The method of claim 21, wherein the parameter comprises a speed of the tip of the first tool.

26. The method of claim 21, wherein the energy is a pulse energy, and the method further comprises:
controlling a repetition rate and pulse duration of the pulse energy.

27. The method of claim 21, further comprising:
varying a pulse repetition rate of the energy between a first pulse repetition rate and a second pulse repetition rate that is higher than the first pulse repetition rate based on a distance between the tip of the first tool and the material.

28. A robotic system, comprising:
a first robotic component configured to control movement of an first device;
a second robotic component configured to control movement of an second device;
a processor; and
a computer-readable memory in communication with the processor and having stored thereon computer-executable instructions to cause the processor to:
insert the first device into a chamber of an anatomical structure;
control movement of the second device within the chamber of the anatomical structure;
control the first device to emit energy to break up material into pieces within the chamber of the anatomical structure;
control the second device to aspirate the pieces of material from the chamber;
control the first robotic component to cause a tip of the first device to travel along a tool path; and
control the first device to gradually vary a parameter of the first device between a first value and a second value based on a distance that the tip of the first device has traveled along the tool path.

29. The robotic system of claim 28, wherein the gradual varying of the parameter is further based on a hardness of the material at the distance along the tool path.

30. The robotic system of claim 28, wherein the gradual varying of the parameter includes compensating for inconsistencies in a speed of the tip of the first device.

31. The robotic system of claim 28, wherein the gradual varying of the parameter is further based on movement of anatomy of the anatomical structure.

32. The robotic system of claim 28, wherein the first device includes an electromagnetic (EM) sensor.

33. The robotic system of claim 28, wherein the parameter comprises a pulse energy of the first device.

34. The robotic system of claim 28, wherein the parameter comprises an effective speed of the tip of the first device.

35. A non-transitory computer readable storage medium having stored thereon instructions that, when executed, cause at least one computing device to:
control movement of an first device within a chamber of an anatomical structure, the first device configured to be coupled to a first robotic component;
control movement of a second device within the chamber of the anatomical structure, the second device configured to be coupled to a second robotic component;
cause the first device to emit energy to break up material into pieces within the chamber of the anatomical structure;
cause the second device to aspirate the pieces of the material from the chamber; and
gradually vary a parameter of the first device between a first value and a second value based on a distance that a tip of the first device has traveled along a tool path.

36. The non-transitory computer readable storage medium of claim 35, wherein the parameter comprises a pulse repetition rate and wherein the gradually varying the pulse repetition rate includes achieving an effective tool tip speed that is substantially equivalent to a speed of the tip of the first device having a consistent pulse repetition rate.

37. The non-transitory computer readable storage medium of claim 35, wherein the parameter comprises a pulse repetition rate of the first device.

38. The non-transitory computer readable storage medium of claim 35, wherein the tool path comprises a spiral and the parameter comprises a spacing between windings of the spiral.

39. The non-transitory computer readable storage medium of claim 35, wherein the first device comprises a plurality of laser fibers arranged to form a shape configured to increase an area that is treated by the energy.

40. The non-transitory computer readable storage medium of claim 35, wherein the first device includes a laser.
